(19) United States
(12) Patent Application Publication
(10) Pub. No.: US 2022/0347015 A1
(43) Pub. Date: Nov. 3, 2022

(54) Vitrector Cutting Device with Distal Illumination Module

(71) Applicant: VisionCare Devices LLC., Anderson, CA (US)

(72) Inventor: Rick M. Morgan, Redding, CA (US)

(73) Assignee: VisionCare Devices LLC., Anderson, CA (US)

(21) Appl. No.: 17/514,421

(22) Filed: Oct. 29, 2021

Related U.S. Application Data
(60) Provisional application No. 63/183,550, filed on May 3, 2021.

(51) Int. Cl. A61F 9/007; A61B 90/30

(52) U.S. Cl. CPC A61F 9/00763; A61B 90/30; A61B 2090/309

(57) ABSTRACT

A vitrector cutting instrument includes a first needle supported by a tool body, the first needle having an inside and outside diameter and a cut window disposed near the distal end, a second needle housed concentrically within the first needle, the second needle chucked in a spring-tensioned needle chuck connected to at least one diaphragm connected by an airline to a switch-controlled pneumatic machine having a control console, the second needle operated from the control console to advance into the cut window and retract from the cut window repetitively defining a cutting process, an illumination module containing at least one light emitting diode (LED) fixed at the distal end of the first needle beyond the cut window, and a power trace extending from the illumination module to a power source. The illumination module may be powered on to aid in illuminating the posterior ocular chamber surrounding the area of cutting.

### CROSS-REFERENCE TO RELATED DOCUMENTS

This patent application claims priority to U.S. provisional patent application serial number 63/183,550 entitled VITRECTOR CUTTING DEVICE WITH DISTAL ILLUMINATION MODULE, filed on May 03, 2021, the disclosure of which in included herein at least by reference.

### BACKGROUND OF THE INVENTION

1. Field of the Invention

The present invention is in the field of medical devices, more particularly surgery devices for operation on the eye and pertains particularly to methods and apparatus for illuminating the posterior ocular chamber during vitrectomy surgery procedures.

2. Discussion of the State of the Art

In the art of vitrectomy surgery, trained optical surgeons use a variety of devices including a vitrector device to perform surgery within the vitreous chamber of the eye. Removing vitreous humor floaters from the internal space in the posterior eye chamber is among a list of common operations performed.

A vitrectomy cutter is a hand-held surgery tool characterized as having at least two hollow small diameter needles, typically an outer needle encompassing a smaller diameter needle that fits concentrically inside the larger needle. The larger needle includes a distal cutting port or window and the smaller needle, which may also include a cutting feature operated by pneumatics to extend into the cutting window of the larger needle and retract therefrom defining one cut operation to break or chop up vitreous humor, typically called floaters in the art.

A vitrectomy cutter device, also referred to as a vitrector device, is connected to a console system that supplies pneumatics in the form of air pressure bursts, vacuum for aspiration, and in some cases injection modality for delivering anesthetics, drugs, or other solutions. The cutting operation may be mitigated with an internal spring and a diaphragm architecture whereby a burst of air against the diaphragm compresses the spring attached to the smaller cutting needle advancing it into the cutting window of the outer needle. The compressed spring immediately recoils after the air burst bringing the smaller needle cutting tip back out of the cutting window placed in the larger needle thus completing one cut cycle. In another case, the vitrector device uses two pneumatic air lines and a diaphragm to deliver two bursts of air to move the inner needle. A first burst pushes the smaller needle cutting tip into the cutting window and a second burst is routed to push the needle back to original position defining one cut cycle.

A typical vitrector device has a cutting speed of about 5,000 cuts per minute but can be lower down to 1,000 or higher up to about 8,000 cuts per minute. In current art, most vitrectomy surgeries utilize at least two separate tools, the vitrector device and a separate end-illumination probe to light up the posterior area of the ocular chamber. Inconveniences in this approach include the requirement of two hands or an assistant to operate the tools simultaneously during surgery. Another inconvenience may be manipulating the light probe constantly to navigate tool shadows that may be cast over the area that requires lighting.

More recently, manufacturers have experimented with optical fibers as a way to illuminate the distal area of a vitrector cutting needle, more specifically the cutting window and areas just beyond in a perimeter cone or beam. Small diameter optic fibers may be run singularly, in tandem, or in small bundles down the outer vitrector needle to illuminate the distil end of the cutter without requiring a separate light probe. Typically, the light is supplied by the console system and radiates through the fiber or fibers to the end of the fibers. However, the materials used may limit the intensity of the light and may result in some glare from the tip of the probe that can interfere with the vision of the surgeon performing the operation. Moreover, there may be safety issues associated with the use of typical illumination types such as halogen, metal halide, and fluorescent type illumination devices.

Therefore, what is clearly needed is an illumination module integrated with the outer needle of a vitrector type device that eliminates or reduces the problems described above.

### BRIEF SUMMARY OF THE INVENTION

According to an embodiment of the present invention, a vitrector surgical cutting instrument is provided including a first needle supported by a tool body, the first needle having an inside and outside diameter and a cut window disposed near the distal end, a second needle housed concentrically within the first needle, the second needle chucked in a spring-tensioned needle chuck connected to at least one diaphragm connected by an airline to a switch-controlled pneumatic machine having a control console, the second needle operated from the control console to advance into the cut window and retract from the cut window repetitively defining a cutting process, an illumination module containing at least one light emitting diode (LED) fixed at the distal end of the first needle beyond the cut window, and a power trace extending from the illumination module to a power source, wherein the illumination module may be powered on to aid in illuminating the posterior ocular chamber surrounding the area of cutting.

In one embodiment, the power source is a rechargeable, switch controlled, battery housed within the tool body. In another embodiment, the power source is a metered alternating circuit (AC) power outlet on the console having a wire connection to the tool body and to the power trace. In one embodiment, the power trace is applied on the outside surface of the first needle and extends longitudinally along the needle connecting at a base of the illumination module. In a variation of this embodiment, the power trace is deposited onto the outside surface of the first needle and is encapsulated along the length of the power trace.

In one embodiment, the illumination module is press fit into the open end of the first needle. In another embodiment, the illumination module is bonded to the open end of the first needle using a cyanoacrylate bonding material and method. In a preferred embodiment, the illumination module includes a translucent dome cover encapsulating the at least one LED. In one embodiment, the at least one LED emits colored light.

In one embodiment, the vitrector surgical cutting instrument further includes at least one fiber optic line culminating at or proximal to the illumination module, the at least one fiber optic line emitting illumination separately and simultaneously with the illumination module. In a variation of this embodiment, the at least one fiber optic line uses an additional power source.

In one embodiment, the illumination module is removably attached to the end of the first needle wherein coupling and uncoupling the illumination module is achieved using a specialized tool. In one embodiment, the first needle and illumination module are permanently adjoined and may be removed from the tool body wherein a replacement first needle with an adjoined illumination module may be selected from a number thereof and installed onto the tool body. In this embodiment, there are intentional differences in available illumination modules adjoined to first needles that may be attributed to variances in dome transparency or dome color and or dome shape. In a variation of this embodiment, the intentional differences in available illumination modules adjoined to first needles may be in the number of LEDs in the illumination module and or intensity ratings relative to intensity of illumination.

In the embodiment having a number of first needles with adjoined illumination modules, a number of first needles including separate illumination modules are provided as part of a kit. In one embodiment wherein the illumination module is domed, the dome color may be amber, green, yellow, or red translucence to aid in identifying medical anomalies in eye tissue. In a variation of this embodiment, the dome may be amber, green, yellow, or red translucence to produce less light intensity in procedures that do not require higher light intensity to successfully perform. In a further variance of the domed illumination module embodiment, the dome includes a reflective material coating over otherwise translucent material for the purpose of reflecting more light angularly shaping light through the dome relative to the longitudinal direction of the inserted probe. In one embodiment, the at least one LED somewhat pivotally attached to a circuit base and therefore angularly adjustable to an outward angle spreading light and to an inward angle to focusing light.

### BRIEF DESCRIPTION OF THE SEVERAL VIEWS OF THE DRAWINGS

FIG. 1 is an elevation view of a vitrector device adapted to host an end-illumination element according to an embodiment of the present invention.

FIG. 2 is a front-end view of the vitrector device of FIG. 1.

FIG. 3 is an enlarged detailed view of detail A of FIG. 1 according to an embodiment of the present invention.

### DETAILED DESCRIPTION OF THE INVENTION

In various embodiments described in enabling detail herein, the inventor provides a unique vitrector cutting implement with an illumination module for illuminating the posterior ocular chamber during a vitrectomy surgery. A goal of the present invention is to combine the efficiency of an end illumination probe with a vitrector type surgery tool. Another goal of the present invention is to provide illumination in the posterior ocular chamber in the vicinity and beyond the cutting window of the outer vitrector needle. It is a further goal of the present invention to provide a type of illumination that is less radiative and therefore less damaging to the eye tissue illuminated.

It is a further goal of the present invention to provide a vitrectomy surgery device with an end illumination module that can be adapted to reduce glare, illuminate at different intensities, and that can be color filtered to both reduce glare and to better identify damaged or diseased eye tissue. The present invention is described using the following examples, which may describe more than one relevant embodiment falling within the scope of the invention. Further goals of the invention include providing an end illumination module that allows the surgeon to illuminate vitreous removal of floaters inside the posterior chamber of the eye and illumination close to the cutting ports of the vitrector for aiding in mitigation of ocular hemorrhaging and various eye traumas in patients.

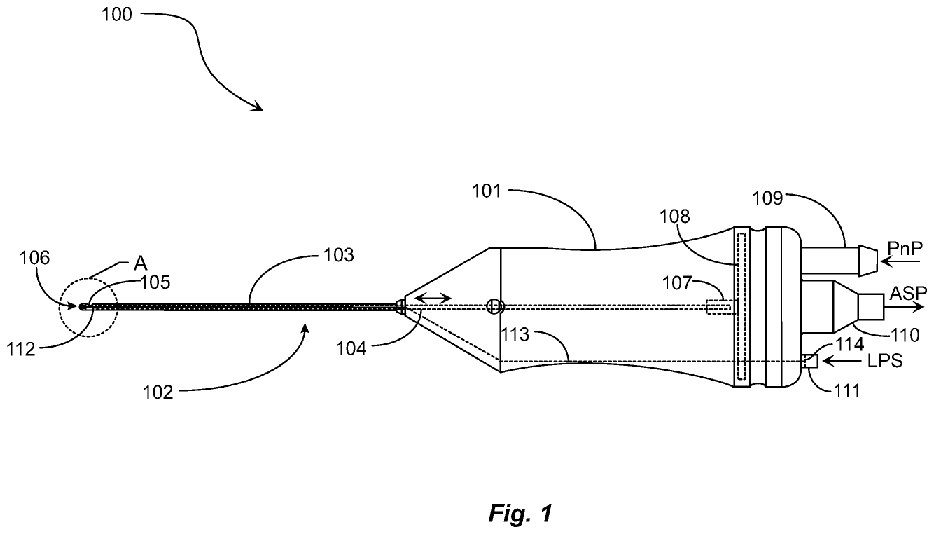

FIG. 1 is an elevation view of a vitrector device 100 adapted to host an end-illumination element according to an embodiment of the present invention. Referring now to FIG. 1, a vitrector surgery device 100 is provided and adapted to host an end-illumination element according to an embodiment of the present invention. Vitrector device 100 is a hand held vitrectomy surgery implement that is routinely used in various procedures required in eye surgery, more specifically for operations conducted in the posterior segment of the eye also referred to in the art as pars plana or a pars plana vitrectomy. Vitrector 100, depicted in elevation view in this embodiment has an ergonomic body 101 that serves as a housing for internal components including at least two vitrectomy needles mounted concentrically referenced herein as probe components 102 including a small diameter inner vitrectomy needle 104 (operable cutting needle), and a larger diameter outer vitrectomy needle 103 fixed, in one embodiment, to the nose cone of body 101.

Vitrector body 101 may be molded from medical-grade plastics or polymer material or other sterilization-able rigid materials. Vitrector body 101 is annular in this example and may be hollow and strategically chambered internally to hold and support internal components and may be airtight and watertight to allow for vacuum through the body and for pneumatic pressure to be used to drive the cutting element or vitrector needle 104. In one embodiment, vitrector body 101 is an assembly wherein the posterior cap end may be removed and the anterior cone supporting needle components 102 may be removable.

Vitrector device 100 has at least two ports disposed at the rear of vitrector body 101. These are an Aspiration Port (ASP) 110, and a pneumatic pressure port (PnP) 109. ASP 110 is disposed at roughly center of the rear of body 101 and provides an egress for chopped vitreous material vacuumed out of the operation area (posterior segment) through the smaller vitrectomy needle 103 and vitrector body 101 and to a collection reservoir (not depicted) associated with the console system (not depicted) that vitrector device 100 has ported connection to during use. PnP 109 is an ingress port for receiving pressurized air bursts from a compressed air or gas source at the console. In this implementation, vitrector device 100 has a third connection to the console adapted as a power port for accepting a power plug to supply power through body 101 and along outer needle 103 to an illumination module broadly defined herein as illumination module 106 disposed at the end of the larger vitrectomy outer needle 103 just past a cutting window 105 disposed near the free end of the needle.

In a preferred embodiment, illumination module 106 includes at least one light emitting diode (LED) set in a power bay at the base of module 106. A power lead circuit trace 112 is applied in this embodiment on the outside surface of outer needle 103 and extends longitudinally along the needle connecting at the base of illumination module 106 to power the at least one LED. In one embodiment, trace 112 is painted on or otherwise deposited on (surface deposition) needle 103 and is encapsulated along the length thereof. An insulated electrical wire 113 may be provided to bridge between trace 112 and a contact pin or plate 114 inside connector 111 (female connector). Electrical wire 113 may be routed through an internal hollow (pathway) in vitrector body 101 provided therein for the purpose.

In one embodiment, outer needle 103 may be anchored at front center of vitrector body 101 while inner needle 104 is much longer extending into the vitrector body to needle chuck 107 adapted to hold the smaller operable needle 104 which is the cutting needle. In one embodiment, air pressure is used to drive a diaphragm 108 (broken boundary) forward toward the distil end of vitrector body 101 and against a centered spring (not illustrated) housed concentrically about the smaller cutting needle 104. Recoil of the spring occurs just after the absence of air pressure to the diaphragm returning the inner cutting needle back to its original position clear of the cutting window.

Cutting needle 104 and fixed needle 103 may be manufactured of high medical grade stainless steel and may exhibit various geometries for representing the cutting interface comprising the cutting window 105 on outer needle 103 and a cutting edge on the free end of inner needle 104. Inner cutting needle 104 extends into body 101 and may be anchored at needle chuck device 107 as previously described. In general practice PnP in the form of quick air bursts hit diaphragm 108 to push needle chuck 107 against the center spring (not shown) advancing the cutting edge of needle 104 into the cutting window 105 on outer needle 103. Spring recoil retracts needle 104 back to an original position defining one cut cycle.

Illumination module 106 provides LED illumination directly at the tip of or end of outer needle 103 just past cutting window 105. Illumination module 106 may illuminate the cutting window 105 on outer needle 103 and an appreciable area past and around the tip of needle 103. Illumination module 106 may be encapsulated in a preferred embodiment.

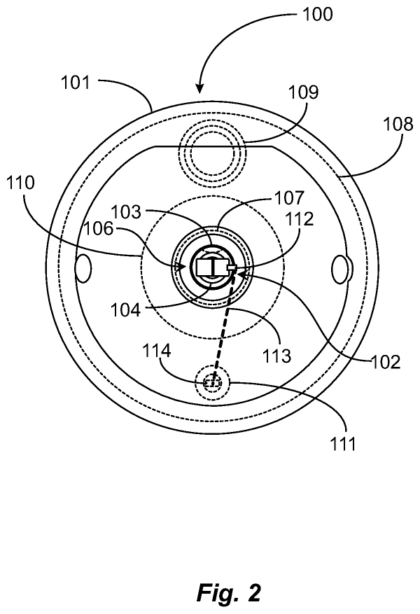

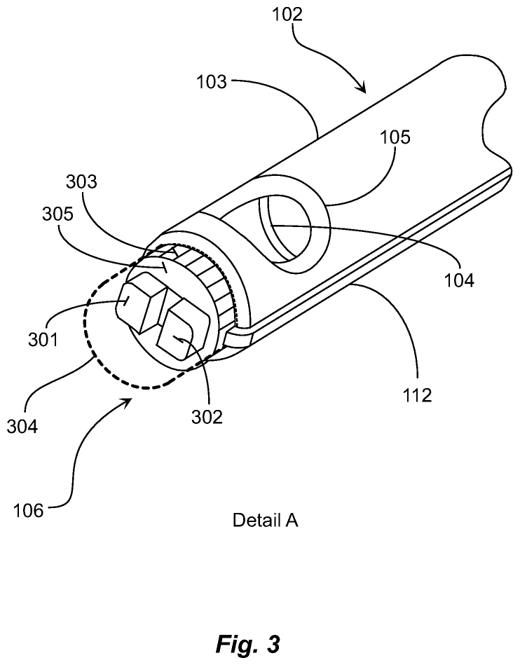

FIG. 2 is a front-end view of the vitrector device of FIG. 1. Referring now to FIG. 2, a front-end view of vitrector device 101 depicts the concentric relationship of probe components 102, more specifically outer needle 103 and inner needle 104 with reference to vitrector body 101 and needle chuck 107 (broken boundary). Illumination module 106 may include one or more LEDs arranged within an annular footprint of the illumination module which may be roughly equal to or smaller than the gauge of the outer needle 103. Two square LEDs are visible in this example and are introduced as elements later in this specification relative to FIG. 3. In this view, PnP 109 and LPS connector port 111 share a vertical center line with ASP 110. This is not required in order to practice the present invention.

In one embodiment without a spring, there are two ingress ports for receiving compressed air bursts, one to advance cutting needle 104 into the cutting window of outer needle 103, and an alternate one for retracting needle 104 back to original position. In one embodiment, vitrector body 101 does not utilize a connector to tap power for the LED illumination module from a console rather a long-lasting chargeable battery may be provided within vitrector body 101 to power the LEDs in illumination module 106. Logically, a charge port may be provided and accessible on the outside of vitrector body 101 in this embodiment. In a variant of the embodiment, vitrector device 100 may have a battery, a charge port, and a power connection plug to an LED power source without departing from the spirit and scope of the invention.

In this example, power trace 112 connects at the side of module 106 extending through the wall thereof to contact, or otherwise connect at a metalized surface (contact point) providing power via electrical contact to base circuitry (not illustrated) powering the one or more LEDs to illuminate them. Wire 113 may connect at the rear portion of outer needle 103 to trace 112 via a rear circuit (not illustrated) or wire lead adapted to be securely to trace 112. Wire 113 may connect at the opposite end to contact plate or lead 114 within power connector (LPS connector) 111. In one embodiment of the present invention, a cyanoacrylate bonding method may be utilized to attach illumination module 106 to the end of outer vitrector needle 103. In another embodiment, illumination module 106 may be press fit into the end of outer needle 103. The forward end of illumination module 106 may include a translucent dome that may cover the one or more LEDs arranged in the module.

Referring now to FIG. 3, an enlarged view of the distal end of probe components 102 referenced within detail A including illumination module 106 of FIG. 1 is depicted according to an embodiment of the present invention. In this implementation, illumination module 106 may be attached to the end of outer needle 103 by press fit method, snap method, or via bonding method. In this embodiment, the diameter of illumination module 106 is smaller than the outside diameter of needle 103 and equal to or just greater than the inner diameter of outer needle 103. In one embodiment, illuminating module 106 may be the same as or larger in diameter than outer needle 103. However, increasing the diameter of illumination module 106 past the outer diameter of needle 103 may require a larger suture after the tool is removed. In a preferred embodiment, illumination module 106 is kept at the same or smaller diameter than the larger gauge of the needle used.

In this embodiment, trace 112 connects to an annular or disc-like circuit base 303 having a substantially flat upper bay surface 305 presenting orthogonal to the longitudinal center of needle 103. Two LEDs are depicted, referenced herein as LED 301 (right) and LED 302 (left), seated side-by-side in bay surface 305 within pocket forms or bays provided for the purpose. In one embodiment, there may be a single larger LED provided, or in another embodiment more than two LEDs without departing from the spirit and scope of the present invention. Likewise, the geometric form of LEDs 301 and or 302 may vary without departing from the spirit and scope of the invention. For example, rather than two LED rectangular elements, a single annular LED element might be provided, or an LED formed as an illumination ring shape. There are many possibilities. Illumination module 106 has a dome structure 304 provided to cover and protect LEDs 301 and 302 from contact with eye tissue. Dome 304 may be fabricated of a clear or transparent medical grade polymer material and may be thermally molded over the outside diameter of circuit base 303 in an airtight manner. In one embodiment, dome 304 is clear and translucent to transparent enabling unfiltered light from LEDs 301 and 302 to pass through to illuminate the posterior chamber during retinal vitrectomy surgery.

In one embodiment, surface wall 305 is transparent or translucent allowing light to pass through the rear of illumination module 106 into the cutting window 105 on outer needle 103. In one embodiment, illumination module 106 is a modular component that may be removably attached to the end of outer needle 103 using a special tool to press into or otherwise couple it to the end of the needle and to pull it from position in the end of the needle. In this embodiment, several different illumination modules might be provided for an operator to select from to attach to the end of the vitrector needle. In this embodiment, differences in available illumination modules may be attributed to variances in dome transparency or color and or shape and number of LEDs, and perhaps intensity ratings relative to intensity of illumination.

In another embodiment, outer needle 103 including a permanently mounted illumination module might be removed from vitrector body and replaced by another outer needle hosting a different permanently mounted illumination module and where a cache of such needles may be provided for an operator to select from. Again, in this embodiment, the difference between the illumination modules may center on dome characteristics or features that an operator may prefer depending upon the procedure the operator is performing including variances in dome transparency or color and or shape and number of LEDs, and perhaps intensity ratings relative to intensity of illumination. For example, dome 304 may be an amber, green, yellow, or red translucency to aid in identifying medical anomalies in eye tissue and in some instances to light intensity in procedures that do not require the highest light intensity to successfully perform.

In still another embodiment, an illumination module 106 may include a dome having some reflective material coated over otherwise translucent material for the purpose of reflecting more light angularly, or perhaps shaping light through the dome relative to the longitudinal direction of the inserted probe. LEDs 301 and 302 may be configured in one embodiment to be somewhat pivotally attached to circuit base 303 as to be somewhat adjustable for example to an angle outward spreading light or to an angle inward to focus light. Domes 304 may be fabricated or treated after fabrication to evenly disperse and diffract and or filter LED light reducing glare.

In some embodiments, the illumination module may emit white LED light. Encapsulation material over the LED module may be different colors for producing specific light color ranges to aid in identification of diseased tissues. LED light may be multicolored. In one embodiment, the illumination module may be disposed within the outer needle disposed at the end. In another embodiment, the illumination module may be positioned over the outer needle like a doughnut type device without departing from the spirit and scope of the invention. In such an embodiment, the illumination module may be positioned on the proximal side of the cutting window in the outer needle.

Illumination module 106 may be used in some embodiments in combination with the use of one or more than one optical fiber that may be bonded to outer needle 103 and that may transmit light from a different source in a console for example, wherein the fiber or fibers stop just short of the cutting window 105 providing two different sources of illumination that may be used simultaneously or independently from one another. In one embodiment using an LED illumination module, there may be two traces like trace 112 and a switch element provided within LPS connector 111 and operable by an operator to select which (if not both or all) of more than one LED in the illumination module receives power. Use of illumination module 106 may reduce or eliminate requirements or suggestions to increase the distance of the illumination element from the retina as may be required with radiative light sources.

It will be apparent to one with skill in the art that the vitrector surgery device of the present invention may be provided using some or all the elements described herein. The arrangement of elements and functionality thereof relative to the apparatus of the invention is described in different embodiments each of which is an implementation of the present invention.

1. A vitrector surgical cutting instrument comprising:
a first needle supported by a tool body, the first needle having an inside and outside diameter and a cut window disposed near the distal end;
a second needle housed concentrically within the first needle, the second needle chucked in a spring-tensioned needle chuck connected to at least one diaphragm connected by an airline to a switch-controlled pneumatic machine having a control console, the second needle operated from the control console to advance into the cut window and retract from the cut window repetitively defining a cutting process;
an illumination module containing at least one light emitting diode (LED) fixed at the distal end of the first needle beyond the cut window;
a power trace extending from the illumination module to a power source;
wherein the illumination module may be powered on to aid in illuminating the posterior ocular chamber surrounding the area of cutting.

2. The vitrector surgical cutting instrument of claim 1, wherein the power source is a rechargeable, switch controlled, battery housed within the tool body.

3. The vitrector surgical cutting instrument of claim 1, wherein the power source is a metered alternating circuit (AC) power outlet on the console having a wire connection to the tool body and to the power trace.

4. The vitrector surgical cutting instrument of claim 1, wherein the power trace is applied on the outside surface of the first needle and extends longitudinally along the needle connecting at a base of the illumination module.

5. The vitrector surgical cutting instrument of claim 4, wherein the power trace is deposited onto the outside surface of the first needle and is encapsulated along the length of the power trace.

6. The vitrector surgical cutting instrument of claim 1, wherein the illumination module is press fit into the open end of the first needle.

7. The vitrector surgical cutting instrument of claim 1, wherein the illumination module is bonded to the open end of the first needle using a cyanoacrylate bonding material and method.

8. The vitrector surgical cutting instrument of claim 1, wherein the illumination module includes a translucent dome cover encapsulating the at least one LED.

9. The vitrector surgical cutting instrument of claim 1, wherein the at least one LED emits colored light.

10. The vitrector surgical cutting instrument of claim 1, further including at least one fiber optic line culminating at or proximal to the illumination module, the at least one fiber optic line emitting illumination separately and simultaneously with the illumination module.

11. The vitrector surgical cutting instrument of claim 10, wherein the at least one fiber optic line uses an additional power source.

12. The vitrector surgical cutting instrument of claim 1, wherein the illumination module is removably attached to the end of the first needle wherein coupling and uncoupling the illumination module is achieved using a specialized tool.

13. The vitrector surgical cutting instrument of claim 1, wherein the first needle and illumination module are permanently adjoined and may be removed from the tool body wherein a replacement first needle with an adjoined illumination module may be selected from a number thereof and installed onto the tool body.

14. The vitrector surgical cutting instrument of claim 13, wherein there are intentional differences in available illumination modules adjoined to first needles that may be attributed to variances in at least one or more of dome transparency, dome color and or dome shape.

15. The vitrector surgical cutting instrument of claim 14, wherein the intentional differences in available illumination modules adjoined to first needles may be in the number of LEDs in the illumination module and or intensity ratings relative to intensity of illumination.

16. The vitrector surgical cutting instrument of claim 13, wherein a number of first needles including separate illumination modules are provided as part of a kit.

17. The vitrector surgical cutting instrument of claim 8, wherein the dome color may be amber, green, yellow, or red translucence to aid in identifying medical anomalies in eye tissue.

18. The vitrector surgical cutting instrument of claim 8, a wherein the dome may be amber, green, yellow, or red translucence to produce less light intensity in procedures that do not require higher light intensity to successfully perform.

19. The vitrector surgical cutting instrument of claim 8, wherein the dome includes some reflective material coating over otherwise translucent material for the purpose of reflecting more light angularly shaping light through the dome relative to the longitudinal direction of the inserted probe.

20. The vitrector surgical cutting instrument of claim 8, wherein the at least one LED somewhat pivotally attached to a circuit base and therefore angularly adjustable to an outward angle spreading light and to an inward angle to focusing light.
